(19) United States
(12) Patent Application Publication
(10) Pub. No.: US 2004/0229292 A1
(43) Pub. Date: Nov. 18, 2004

(54) Use of FGF-18 in the diagnosis and treatment of memory disorders

(76) Inventors: Sebastiano Cavallaro, Tremestrieri Etneo (IT); Velia D'Agata, Tremestrieri Etneo (IT); Franck Dufour, Orsay Cedex (FR); Daniel L. Alkon, Bethesda, MD (US)

Correspondence Address: MILBANK, TWEED, HADLEY & MCCLOY LLP, INTERNATIONAL SQUARE BUILDING, 1825 EYE STRET, N.W. #1100, WASHINGTON, DC, 20006, US

(21) Appl. No.: 10/720,091

(22) Filed: Nov. 25, 2003

Related U.S. Application Data
(60) Provisional application No. 60/429,321, filed on Nov. 26, 2002.

(51) Int. Cl. G01N 33/53; G01N 33/567; A61K 38/18

(52) U.S. Cl. 435/7.2; 514/12

(57) ABSTRACT

The invention provides for the use of Fibroblast Growth Factor-18 (FGF-18) to improve learning and memory. The invention also relates to methods of measuring expression levels of FGF-18 in the central nervous system, and the use of such measurements for diagnostic purposes. The invention is expected to be useful in the areas of associative learning, consolidated memory, drug development and analysis, and in the treatment of certain diseases associated with impaired hippocampal function such as dementia due to Alzheimer's Disease.

### CROSS-REFERENCE TO RELATED APPLICATIONS

This application claims priority to provisional application 60/429,321 filed Nov. 26, 2002.

### FIELD OF THE INVENTION

The invention relates to the fields of gene expression measurement, spatial learning, and memory, to fibroblast growth factors, and to the diagnosis and treatment of diseases associated with impaired function of the hippocampus such as dementia due to Alzheimer's disease.

### BACKGROUND OF THE INVENTION

For more than a century, two forms of memory have been distinguished by their duration: short-term memory (STM), which is rapidly formed and can outlast training for minutes or hours [1], and long-term memory (LTM), which can last from hours to days, weeks or even years [2]. In both vertebrates and invertebrates, STM is based on transient modification of preexisting molecules, capable of rapidly altering the efficacy of synaptic transmission. In contrast, LTM is based on changes in synaptic efficacy (long-term potentiation, or LTP), a relatively long-lived increase in synaptic strength produced by the restructuring of synapses [3]. LTM can be blocked by inhibitors of transcription or translation, indicating that it is dependent on altered gene expression and/or de novo gene expression. Proteins newly synthesized during memory consolidation may contribute to restructuring, enhancing the duration of short-term memory.

The morphological restructuring of synapses in LTM is thought to be effected by mechanisms similar to those used for brain development. Fibroblast Growth Factors (“FGF”) are among the proteins that play vital roles in controlling embryonic development, cell growth, morphogenesis, and tissue repair in animals [4]. FGF-18 is one member of this family of proteins; it is a peptide consisting of 207 amino acids, encoded by a single memory related gene associated with spatial learning. It is expressed primarily in the lungs and kidneys and at lower levels in the heart, testes, spleen, skeletal muscle, and brain [5]. Sequence comparison studies indicated that FGF-18 is highly conserved between humans and mice and is most homologous to FGF-8 among the FGF family members. In continuing studies investigating the full role of FGF-18 in cellular and tissue development, FGF-18 has thus far been identified as a signaling molecule for proliferation in the adult lung and developing tissue, and it has been linked to cancerous cells. However, its influential role in hippocampal regulatory pathways related to memory was not known prior to the present invention.

Each cell within an organism contains all information required to produce any bodily protein. This information is stored as genes within the organism's DNA genome. The number of human genes is estimated to be approximately 100,000 but only a portion are actually present as proteins [6]. Some proteins serve functions necessary for each cell. Other proteins serve specialized functions only required in specific cell types. Since a cell's specific function is mainly determined by the proteins expressed, the transcription process of gene conversion into mRNA, and subsequent translation into protein, is highly regulated and to a large extent directs cellular activity.

Since the quantity of a particular protein is often reflected by the abundance of its mRNA, a variety of methods have been used to identify a limited number of mRNAs differentially expressed during the formation of LTM. Increased or, less often, decreased transcription of genes has been demonstrated during specific time windows following learning. These prior art approaches were focused on individual genes or genetic pathways, and failed to address the massively parallel nature of genome activities and the collective behavior of the genes that ultimately control the molecular mechanisms underlying brain functions.

The hippocampus plays a crucial role in learning processes and certain types of memory. Individuals who lose hippocampal function retain memory for events that occurred prior to the loss and only have immediate memory, lasting less than a few minutes, for all events after the loss (anterograde amnesia). Thus, the hippocampus is thought to interpret the importance of incoming sensory information and to determine what input is worth remembering; it then transmits signals that make the mind rehearse the information over and over again until permanent storage takes place.

Numerous studies of the effects of ablation of the hippocampal of rodent, primate, and other non-human species have been conducted. Memory disorders and spatial performance are associated with hippocampal function. Morphological changes in the hippocampus, including cell loss, is associated with epilepsy, schizophrenia, Alzheimer's disease, and Huntington's disease [7]. Research data from animals show glucocorticoids secreted during stress can damage the hippocampus and impair the ability of the hippocampus neurons to survive neurological insults [8]. Sustained high levels of glucocorticoids may damage the hippocampus in humans as well; patients with Cushing syndrome reportedly suffer hippocampal atrophy proportional to hypersecretion of glucocorticoids. It is well-established that proteins that regulate the cell cycle in yeast, nematode, fly, rate, and man have common chemical or structural features and modulate the same general cellular activity. Consequently, animal model systems are of great value for testing medical hypotheses for development and testing of diagnostic and therapeutic agents for human conditions, diseases, and disorders.

Due to the broad variety of genes and cross talk of gene pathways involved in controlling the molecular mechanisms underlying brain function, only gene expression profile analysis properly analyzes the complete regulatory pathway of the genes exhibiting such control. This analysis enables genes to be grouped into distinct clusters that correlate with major cellular development and regulatory events [9]. Gene expression profiling for a given cell or tissue quantitatively converts the 3′ region of mRNA upstream from the polyadenylated stretch in mRNA into cDNA. This provides an accurate representation of the molar composition of mRNA [1] Genes constitutively expressed in the hippocampus of untrained rats have been analyzed by Kaser et al. [10]. but differential expression associated with learning was not reported. Gene expression levels may be perturbed by experimental or environmental condition(s) associated with a biological system such as exposure of the system to a drug candidate, the introduction of an exogenous gene, the deletion of a gene from the system, or changes in cultural conditions. Comprehensive measurements of gene and protein expression profiles and their response to perturbation have a wide range of utility, including the ability to compare and understand the effects of drugs such as FGF-18 as well as to diagnose disease, and optimize patient drug regimens.

The Morris water maze is a widely accepted method of measuring hippocampal learning and memory performance [11]. It consists of a water pool with a hidden escape platform where the subject must learn the location of the platform using either contextual or local cues. By combining physical challenges with visual cues, rats are encouraged to navigate themselves through the water maze to locate a hidden platform that enables them to escape from the water. Performance is videotaped and computer-assisted image analysis is used to measure predetermined variables, such as time and distance traveled. These measurements generate data that provide insight into the learning ability, memory, and spatial learning of the animal tested. Performance in the Morris Water Maze relies on several mechanisms, including attention, learning and memory, vision and motor coordination. The cognitive processes that underlie performance in this test are thus dependent on many biochemical pathways.

Once spatial learning and memory are assessed using the Morris water maze, microarray technology may be used to quantitate the expression level of large numbers of mRNA transcripts simultaneously. This technique provides the ability to monitor the expression level of a large number of mRNA transcripts at one time [12], and it has been used to examine differences in hippocampal gene expression between mouse strains that perform well on the Morris water maze and strains that perform poorly [13]. Efforts to discover genes differentially expressed in water-maze trained rats, using RNA fingerprinting, have been reported as well. [14].

Microarray technology is a hybridization-based process that allows simultaneous quantitation of many nucleic acid species by tagging mRNA representations with different fluorescent tags that emit a different color light. This technique immobilizes small amounts of pure nucleic acid species on a glass surface, hybridizes them with multiple fluorescently labeled nucleic acids, and then detects and quantitate the resulting fluor-tagged hybrids with a scanning confocal microscope. The entire process can be very highly automated. When used to detect transcripts, a particular RNA transcript (an mRNA) is copied into DNA (a cDNA). This copied form of the transcript is then immobilized on a glass surface. The entire complement of transcript mRNAs present in a particular cell type is extracted from cells and then a fluor-tagged cDNA representation of the extracted mRNAs is made by reverse-transcription, an in vitro enzymatic reaction. Fluor-tagged representations of mRNA from several cell types, each tagged with a fluor emitting a different color light, are hybridized to the array of cDNAs and then fluorescence at the site of each immobilized cDNA is quantitated. This analytic scheme is particularly useful for directly comparing the abundance of mRNAs present in two different cell types.

Measurements of cellular levels of gene expression, mRNA abundance, and protein expression provide a wealth of information about a cell's biological state. These levels are known to change in response to drug treatment and other perturbations of the cell's biological state, and they are generally collectively referred to as the “profile” of the cell's biological state. Due to the complexity of these cellular processes, profile measurements of a particular cell or tissue are typically determined before and after the biological system has been subjected to a perturbation, and attention is given to changes in the profile due to the perturbation. Such perturbations include experimental or environmental condition(s) associated with a biological system such as exposure of the system to a drug candidate, the introduction of an exogenous gene, the deletion of a gene from the system, or changes in cultural conditions. Comprehensive measurements of profiles of gene and protein expression and their response to perturbation have a wide range of utility, including the ability to compare and understand the effects of drugs such as FGF-18, diagnose disease, predict susceptibility to disease, and optimize patient drug regimens.

### SUMMARY OF THE INVENTION

Applicants have discovered that expression of Fibroblast Growth Factor 18 (FGF-18) in the brain is increased when an animal is engaged in spatial learning, and have discovered that administration of exogenous FGF-18 significantly enhances the performance of animals in the Morris water maze test. In a first aspect, the invention provides a method of enhancing learning and memory consolidation in an animal, which comprises administering an effective amount of FGF-18.

Deficits in memory-related gene products are known to be associated with learning deficits. For example, deletion of the CaM kinase IIα gene from mice impairs their performance in the water-filled Morris maze [15]. Similar deficits are induced by knocking out genes for tyrosine kinase Fyn [16] and a hippocampal NMDA glutamate receptor [17,18]. FGF-18 is a memory- and learning-enhancing factor that is upregulated during leaming, and accordingly a deficiency in FGF-18 is expected to be associated with a deficiency in learning. The invention thus provides a method of diagnosing memory disorders, or for identifying a predisposition to such disorders, by quantitation of FGF-18 (FGF-18) gene expression in the hippocampus.

The invention also provides for the use of FGF-18 to facilitate learning and memory, and to treat subjects suffering from impaired learning and/or memory functions.

The invention also provides a method for screening for drugs that modulate FGF-18 gene expression, and in another aspect the invention provides methods for discovering therapeutic target proteins for pharmacological intervention, and methods for drug discovery based upon the information provided by gene expression analysis.

The invention provides a method of enhancing memory, attentive cognition or learning comprising the administration of a composition, wherein the composition comprises an effective amount of FGF-18 and a pharmaceutically acceptable carrier, to a subject in need thereof. In a preferred embodiment, the subject suffers from a condition selected from the group consisting of: impaired cognitive performance, learning deficit, cognition deficit, attention deficit, epilepsy, schizophrenia, Alzheimer's disease, and amnesiac syndromes.

The present invention also provides a method for the administration of a composition, wherein the composition comprises an effective amount of FGF-18 and a pharmaceutically acceptable carrier, to a subject in need thereof, wherein the composition is administered in an amount effective to increase FGF-18 levels in the subject's brain. In a preferred embodiment, the composition is administered in an amount effective to increase FGF-18 levels in the subject's hippocampus.

The present invention also provides for the use of FGF-18 for the production of a medicament for the improvement of attentive cognition, the improvement of memory or for the improvement of learning. In a preferred embodiment, the present invention provides for the use of FGF-18 for the production of a medicament for the treatment of a subject suffering from a condition selected from the group consisting of: impaired cognitive performance, learning deficit, cognition deficit, attention deficit, epilepsy, schizophrenia, Alzheimer's disease, and amnesiac syndromes.

### BRIEF DESCRIPTION OF THE FIGURES

FIG. 1 outlines the process for identifying memory related genes.

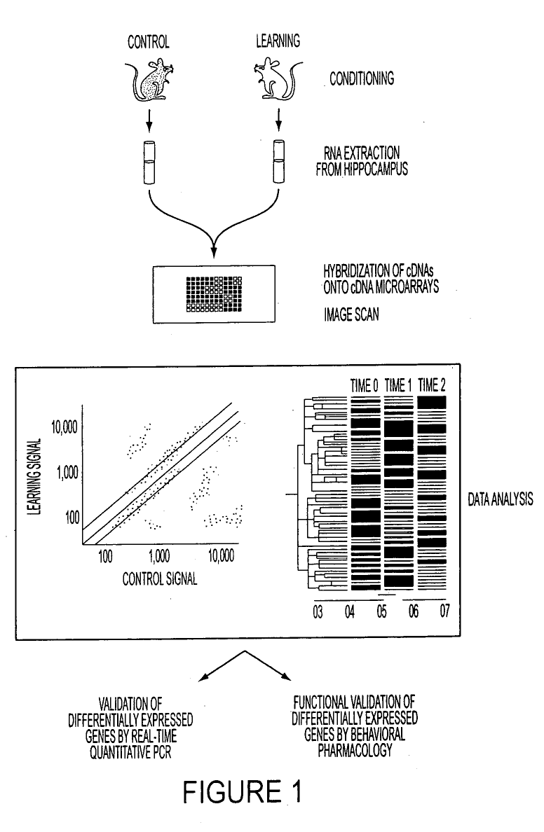

### DETAILED DESCRIPTION OF THE INVENTION

The invention provides a method for determining the susceptibility of a subject to a condition associated with impaired hippocampal function. Such conditions include but are not limited to impaired cognitive performance, learning deficit, cognition deficit, attention deficit, epilepsy, schizophrenia, Alzheimer's disease and amnesiac syndromes. The method comprises the steps of: (a) obtaining from the central nervous system of the subject an mRNA-containing sample which comprises mRNA encoded by the Fibroblast Growth Factor-18 gene, and (b) quantitating the Fibroblast Growth Factor-18 mRNA in the sample. The level of the Fibroblast Growth Factor-18 mRNA is indicative of the subject's susceptibility to one or more conditions associated with impaired hippocampal function. Preferably, the mRNA-containing sample is obtained from the hippocampus.

Methods of quantitating the FGF-18 mRNA include but are not limited to Northern blotting, nuclease protection assays, array hybridization, RT-PCR (reverse transcription-polymerase chain reaction), and hybridization with directly- or indirectly-labeled oligonucleotide probes (e.g. biotin and digoxigenin labeled probes with enzyme-coupled avidin or antibody). Such methods are well-known to those skilled in the art.

The invention also provides a method for determining the pharmacological effect of a compound on the level of FGF-18 gene expression, comprising the steps of: (a) growing one or more cultures of neural cells, preferably human neural cells, (b) measuring the level of FGF-18 gene expression in the cultured cells, (c) contacting the compound with at least one of the cultures of neural cells, and (d) measuring the level of FGF-18 gene expression in the cultured cells that have been contacted with the compound. In this method, a difference in the gene expression level associated with contact of the cultured cells with the compound is indicative of a pharmacological effect of the compound. Methods of neural cell culture are well-known in the art [19].

More generally, the invention provides a method of identifying compounds likely to have a pharmacological effect on learning, memory, and/or memory consolidation, by a process comprising the steps of (a) identifying a memory-related gene that is differentially expressed in the brains of animals that have learned a task, relative to animals that have not learned the task, (b) growing one or more cultures of neural cells, preferably human neural cells, (c) measuring the level of expression of the gene identified in step (a) in the cultured cells, (d) contacting a compound to be tested with at least one of the cultures of neural cells, and (e) measuring the level of expression of the gene identified in step (a) in the cultured cells that have been contacted with the compound. In this method, a difference in gene expression level associated with contact of the cultured cells with the compound is indicative of a pharmacological effect of the compound.

The methods set forth above in connection with measurement of FGF-18 gene expression levels may be employed in the measurement of differential gene expression of other memory-related genes. In the methods above, the preferred method of measuring gene expression levels is through hybridization of transcripts to a polynucleotide microarray.

The invention also provides a method of treating a condition associated with memory impairment, including but not limited to impaired cognitive performance, learning deficit, cognition deficit, attention deficit, epilepsy, schizophrenia, Alzheimer's disease, and amnesiac syndromes, which comprises the step of administering to an individual in need of such treatment a therapeutically effective amount of Fibroblast Growth Factor-18.

The invention also provides a method for identifying memory-related proteins, which serve as potential targets for pharmacological intervention in the treatment of conditions associated with memory impairment. The method comprises the steps of (a) providing naive, swimming control, and water-maze trained animals; (b) extracting mRNA from the hippocampus of the naive, control and trained animals; (c) determining differential gene expression levels by measuring and comparing mRNA levels in naïve, control and trained animals so as to identify “memory related genes”; and (d) quantitating protein levels reflecting “memory related genes” for both control and target groups. In a preferred embodiment of the invention, mRNA levels are measured by reverse-transcribing the extracted mRNA, and hybridizing the resulting cDNA to a microarray. The differentially expressed genes quantified in step (d) may be validated by quantitative RT-PCR and behavioral pharmacology.

Identification of memory-related protein, and memory-related genes, is accomplished in the present invention by relating mRNA induction or suppression to a learning task. The mRNAs that are induced or suppressed may be related to the genes encoding the mRNAs, and thence related to the encoded proteins.

To relate mRNA induction or suppression to a learning task, rats were trained for four consecutive trials to locate a submerged island in a water maze. The rats completed the task within 2.56±0.49 min (mean±SD) and their latency time to find the island was reduced from 47.8±11.3 sec to 26,3±6.9 sec, indicating that the rats had indeed learned the task. Swimming control rats were allowed to swim in the pool in the absence of the island for 2.5 min. To verify that the trained rats in fact learned the spatial location of the island, a group of six rats was trained to find the island and tested 24 h later on a quadrant analysis. The trained rats swam significantly longer in the quadrant where the island was located (36.5%±3.2% of the total distance compared with 22.5%±2% and 21.8%±2.9% in the two adjacent quadrants and 19.1%±4.1% in the opposite quadrant; ANOVA P<0.01).

Hippocampal gene expression profiles in naïve, swimming control, and water-maze trained animals were measured by using microarrays containing more than 1,200 genes relevant to neurobiology. When gene expression profiles in the naïve and swimming control animals 1, 6, and 24 h after swimming sessions were compared, 345 genes (27.3%) were found to be differentially expressed more than 2-fold in at least two of the four conditions. These genes, operationally defined as “physical activity-related genes” (PARGs) indicate that physical activity and mild stress associated with behavioral training has a significant impact on hippocampal gene expression.

When gene expression levels in swimming control animals were compared with water-maze-trained animals 1, 6 or 24 h after training, 140 genes (11%) were found to be differentially expressed and were operationally defined as “memory-related genes” (MRGs). The majority of these MRGs (110 of 140), were also PARGs, i.e., influenced by physical activity. Among MRGs, 91 genes were down-regulated in the hippocampus of water maze-trained animals, whereas 55 genes were up-regulated.

A hierarchical clustering method was used to group memory related genes on the basis of similarity in their expression patterns. Genes represented by more than one probe set on the array, such as inducible nitric oxide synthase, inositol 1,4,5-triphosphate receptor type 1, microtubule-associated protein 2, and Ca²⁺/calmodulin-dependent protein kinase IIα were clustered next to, or in the immediate vicinity of each other, indicating that the effects of experimental noise or artifact are negligible. Although no information on the identity of the samples was used in the clustering, in some cases genes segregated according to their common biological functions. For example, genes encoding for membrane trafficking proteins, such as synaptotagmins 7 and 8, or syntaxin 2, 5, and 8, and most of the genes encoding for γ-aminobutyric acid (GABA) A and B type receptors were expressed concordantly. The most evident trait of the clustered data was that MRGs showed entirely different temporal patterns of expression in swimming control vs. water maze-trained animals.

Although the data obtained represented the average gene expression from two separate microarray analyses performed on pooled hippocampal RNA samples from naïve, swimming control, and water maze-trained animals, there could be differences in gene expression between individual animals. To address this question and to confirm the reliability of the array data 15 genes were selected and their differential expression in the hippocampal mRNA of individual animals was quantitatively validated by using real-time quantitative RT-PCR. Remarkably, the pattern of gene expression from sample to sample observed in the microarrays closely paralleled the pattern observed using real-time RT-PCR. The minimum and maximum correlation coefficients between the two profiles were 0.72 and 0.99, respectively.

Fibroblast growth factor (FGF)-18 was the only MRG not influenced by physical activity that was increased 1, 6, and 24 h after water maze training. To explore the effect of FGF-18 in spatial leaming, the effect of a single exogenous dose of FGF-18 on spatial learning were determined. Adult male rats were trained in a Morris water maze for two trials and then injected intracerebroventricularly with 0.94 pmol of FGF-18 or vehicle. As shown in Table 1, animals treated with FGF-18 displayed significantly improved spatial learning behavior (P<0.05) compared with vehicle-injected control animals. FGF-18 treatment induced a 49% reduction in the escape latency, but no significant changes in motor activity.

TABLE 1
Effects of exogenous FGF-18 on water maze learning.

| | Latency, sec | | Distance, m | |
|---|---|---|---|---|
| Treatment | Day 1 | Day 2 | Day 1 | Day 2 |
| Control | 48.2 ± 16.1 | 37.1 ± 11.1 | 16.5 ± 4.3 | 10.4 ± 3.1 |
| FGF-18 | 46.6 ± 16.7 | 19.2 ± 6.3*† | 15.2 ± 5.7 | 6.1 ± 2.0* |

*Day 1 vs. Day 2 P < 0.05,
†control vs. FGF-18, P v 0.05

The results show that both learning and physical activity have profound effects on hippocampal gene expression. Most of the MRGs, those differentially expressed between the swimming and spatial learning animal groups, were also affected during swimming alone, but with entirely different temporal patterns of expression as shown in the clustered data. Although learning and physical activity involve common groups of genes, the behavior of learning and memory can be distinguished from unique patterns of gene expression across time.

All of the MRGs identified have a recognized function and can be classified into six major groups based on their translated product: (i) cell signaling, (ii) synaptic proteins, (iii) cell-cell interaction and cytoskeletal proteins, (iv) apoptosis, (v) enzymes, and (vi) transcription or translation regulation, described in more detail below.

Some of these genes have been previously related to synaptic plasticity, memory, or cognitive disorders, whereas others provide a significant number of unique entry points that have not been recognized previously. The exact role and functional relationships of some of the genes and proteins implicated are also yet to be recognized. For this reason, only some of the MRGs implicated by microarray analysis are discussed herein. As more time points, behavioral paradigms, and pathophysiological conditions are used for similar studies, and more complete high-density arrays become available, a more complete interpretive framework will emerge as to the key genes and pathways underlying learning and memory.

(i) Cell Signaling. The group of genes involved in cell signaling is the largest and includes a subgroup of neuropeptides, growth factors, and their receptors. Among them is FGF-18, a member of the FGF family, which has been shown to stimulate neurite outgrowth [20]. Although the function of this peptide is still unknown, the other members of its family are important signaling molecules in several inductive and patterning processes, and act as brain organizer-derived signals during the formation of the early vertebrate nervous system. The expression of FGF-18 was induced by water maze training but not physical activity. This result, together with the ability of FGF-18 to enhance spatial memory when exogenously administered, is strong evidence in favor of its involvement in learning and memory.

Differential expression of interleukin-1β (IL-1β), interleukin 15 (IL-15), and interleukin-2 (IL-2) receptor a chain suggests a physiological role of brain cytokines in memory consolidation processes. Indeed, the reduction of IL-1β mRNA in water maze-trained animals is consistent with previous studies showing that central II-1β administration and agents that induce central IL-1β activity impair the consolidation of memories that depend on the hippocampal formation.

Enhanced expression of corticotropin-releasing hormone in water-maze-trained animals is consistent with evidence obtained in another learning paradigm [21].

The subgroup of G protein-coupled receptors includes two GABA B-type receptor splice variants, GABA_{B1d} and GABA_{B2a}. Functional GABA_{B} receptors, whose function depends on dimerization of GABA_{B1} and GABA_{B2}, are known to activate second messenger systems and modulate potassium and calcium channel activity, thereby controlling the presynaptic transmitter release and the postsynaptic silencing of excitatory neurotransmission. GABA_{B} receptor agonists or antagonists are known to impair or facilitate, respectively, cognitive performance in the Morris water maze tasks as well as other kinds of learning [12]. By reducing GABA_{B} receptor signaling, the down-regulation of GABA_{B1d} and GABA_{B2a} 1 hour after water maze training may exert a mnemonic effect similar to that produced by GABA_{B} receptor antagonists.

Dopamine 1A and D4 receptors are down- and up-regulated, respectively, 1 hour after water maze training. These receptors are coupled to different G proteins and their change in expression may allow for the modulation of a neuronal dopamine-mediated signal.

The opioid receptor-like receptor is decreased 1 hour after water maze training. This receptor is a G protein-coupled receptor structurally related to the opioid receptors, whose endogenous ligand is the heptadecapeptide nociceptin, which has been implicated in sensory perception, memory process, and emotional behavior [23, 24].

The adenosine receptor A1, which is negatively coupled to adenylate cylase, decreased 1 hour after water maze training. Adenosine is thought to exert a tonic inhibitory role on synaptic plasticity in the hippocampus [25]. Its decrease, therefore, may exert a facilitative role during learning and memory.

The insulin receptor was increased in swimming control and decreased in water maze-trained rats, whereas the precursor of its endogenous ligand, insulin, was detectable only 24 h after water maze training. The fine balance of brain insulin and its receptor may regulate cognitive functions [26].

The subgroup of ligand-gated ion channels include five GABA_{A} receptor subunits which were all differentially expressed 1 hour after water maze training. Four of them, α4, α5, β2, and γ2, where down-regulated, whereas one, the π subunit, was up-regulated. Changes in the expression of specific GABA_{A} receptor subunits may affect the composition and pharmacology of GABA_{A} receptor assemblies. These changes may also be relevant in consideration of the vast number of drugs such as anxiolytics, anticonvulsants, general anesthetics, barbiturates, ethanol, and neurosteroids, which are known to elicit at least some of their pharmacological effects through GABA_{A} receptor subunits [27].

The expression of glutamate ionotropic receptors is dynamically regulated during spatial learning. N-methyl-D-aspartic acid receptor (NMDA-R) 1, which possesses all properties characteristic of the NMDA receptor-channel complex, is down-regulated 1 hour after water maze training, whereas NMDA-R2A, which has regulatory activities, is up-regulated after 24 h. One 1-α-amino-3-hydroxy-5-methyl-4-isoxazolepropionate (AMPA) receptor α3 subunit is down-regulated 1 hour after training. Two kainite receptors, GluR6 and GluR5-2, are up-regulated 6 and 24 h, respectively, after training. Plastic changes of different combinations of glutamate receptors night have profound effects on glutamate responsiveness [28].

The subgroup of ion channels includes several proteins that play a role in the maintenance of ionic homeostasis. Among these are ten potassium (K⁺) channel subunits: two Shaker (Kcna5 and Kcna5), two Shab (Kcnb1 and Kcnb2), one Shal (Kcnd2) and one EAG-related (Kcnh5) voltage-dependent K⁺ channel subunits' one Ca²⁺-activated (Kenn2) and three inwardly rectifying (Kcjn4, Kcjn11, and Kcjn6). Expression changes of different K⁺ channel subunits may alter the composition of the channel complexes and would affect cellular excitability [29]. Although the exact contribution of each of the above subunits during spatial memory is unknown, seven of the ten are down-regulated after water maze training and may produce increased excitability.

The subgroup of proteins involved in intracellular signaling includes several proteins involved in the intracellular homeostasis of calcium, sodium, and potassium ions. Among these is the frequenin homolog, also known as neuronal calcium sensor-1, which has recently been shown to regulate associative learning [30].

The subgroups of proteins involved in neurotransmitter transport includes GABA, glutamate, and serotonin transporters. The GABA and glutamate transporters are down-regulated 1, 6, or 24 h after water maze training, whereas the serotonin transporters is up-regulated after 1 hour. Neurotransmitter uptake by nerve terminals and glial cells is crucial for providing a reservoir of transmitter or transmitter precursors and the termination of synaptic events [31]. Changes in the expression of these transporters, therefore may have profound effects on neurotransmission by controlling neurotransmitter levels at the synaptic cleft.

The subgroup of signaling enzymes includes a number of proteins previously implicated in learning and memory. After water maze training, a strong induction of the inducible form of nitric oxide synthase (Inos) was observed. This enzyme produces nitric oxide (NO), a molecule involved in neurosynaptic transmission, and is induced in many pathological conditions. Although the role of NO is learning and memory is still unclear, some studies have reported that systemic NO inhibition has deleterious effects in water maze learning [32,33,34] and in learning in Aplysia [35]. The role of iNOS in the hippocampus, therefore, may go beyond its well-established detrimental function in neurological disorders and could contribute to the mechanisms underlying learning and memory.

Two genes encoding enzymes involved in the mitogen-activated protein kinase (MAPK) signaling cascade, p38 MAPK and MAPK phosphatase, were found to be differentially expressed after water maze training. This signaling cascade has been previously implicated in the development of synaptic plasticity underlying learning and memory [36,37,38]. However, there are three subfamilies of MAPKs that are activated by different upstream cascades and are involved in the regulation of distinct nuclear transcriptional factors [39]. As suggested by the present observations and previous studies [40], long-term memory may involve different MAPKs and/or their MAPK phosphatase.

Differential expression of Ca²⁺/calmodulin-dependent protein kinases, belonging to a class of signaling enzymes extensively implicated in memory formation and consolidation [41], was observed after water maze training.

Other proteins involved in signal transduction include Ania-3, a short form of the Homer family of proteins which bind to group 1 metabotropic glutamate receptors, inositol triphosphate receptors, ryanodine receptors, and NMDA receptor-associated Shank proteins and have been implicated in synaptogenesis, signal transduction, receptor trafficking, and axon pathfinding [42]. The long Homer forms are constitutively expressed and self-associate to function as adaptors to couple membrane receptors to intracellular pools of releasable Ca²⁺. The short Homer forms compete with the long Homer proteins for binding to signaling components, thus functioning as endogenous dominant-negative regulators of receptor-induced Ca²⁺ release from intracellular stores. Down-regulation of Ania-3 in water-maze-trained animals may modulate the properties of the long Homer forms and be involved in activity-dependent alterations of synaptic structure and function.

Up-regulation of another signaling molecule, citron, was found 24 h after water maze training. Citron is a neuronal ρ-target molecule associated to the postsynaptic scaffold protein PSD-95, which plays an important role in the anchoring and clustering of neurotransmitter receptors at the synapses [43]. The expression of citron may provide a cross talk between the ρ signaling pathway, which has been implicated in the mechanisms of neuro-plasticity, and in neurotransmitter receptors such as the NMDA receptor.

(ii) Synaptic Proteins. The group of synaptic proteins includes a number of proteins that regulate membrane trafficking and fusion. They include synaptojanin 1, four members of the syntaxin family of proteins (syntaxin 2, 5, 8, and 12), five synaptotagmins (2, 4, 5, 7, and 8), and synaptosomal-associated protein-25. Different expression of these proteins, which are involved in different steps of membrane trafficking and fusion [44], may regulate synaptic plasticity by affecting cellular functions such as secretion, endocytosis, and axonal growth.

(iii) Cell-Cell Interactions and Cytoskeletal Proteins. The group of cell-cell interactions and cytoskeletal proteins includes a vast number of proteins whose change in expression may reflect the morphological adaptation of brain cells during formation of memory. Among them, for example, is δ-catenin, a component of the cell-cell adherens junctions expressed specifically in the nervous system. δ-catenin is down-regulated during neuronal migration and expressed in the apical dendrites of postmitotic neurons [45]. Changes in δ-catenin expression, therefore, are considered to be fundamental for the establishment and maintenance of dendrites and synaptogenesis. δ-Catenin was originally discovered as an interactor with presenilin 1, whose mutation causes early-onset familial Alzheimer's disease. In addition, hemizygosity of δ-catenin is associated with severe mental retardation in the cri-du-chat syndrome that is associated with severe mental retardation [46].

The hippocampal expression of several proteins involved in microtubule formation was reduced 1 hour after water maze training. Among these are β-tubulin, neuraxin, and microtubule-associated proteins 2 (MAP2) and 5 (MAP5). The reduced expression of MAP2, in particular, was confirmed in three redundant probe sets. Altered expression of MAP2, which is critical for dendritic stability, has been shown with contextual memory, long-term potentiation, aging, epilepsy, Alzheimer's disease, and Rett syndrome [47,48,49,50,51,52]. We have recently found altered expression of MAP2 in a transgenic animal model of fragile X syndrome [53], which shows behavioral deficits in the Morris water maze [54]. Expression of several others proteins involved cell-cell and cell-matrix interactions was found to be increased (intercellular adhesion molecule-1, C-CAM2a isoform) or more often decreased (neurexin 1, connexin 43, contactin 1, chondroitin sulfate proteoglycan 3, myelin-associated glycoprotein, and axonal glycoprotein). Call adhesion molecules have already been implicated in synaptic plasticity, learning, and memory [55]. Together, their changes may be critical in regulating cell-cell recognition and the establishment of mature dentritic relationships in the neuropil.

(iv) Apoptosis. The group of proteins involved in apoptosis includes Bcl-2-related death gene product BOD-L, caspases 1 and 6, and DP5, which are all up-regulated after water maze training. In agreement with other studies [56], our date suggest that beyond their roles in cell death, apoptotic and anti-apoptotic cascades may play roles in synaptic plasticity.

(v) Enzymes. The group of enzymes includes two proteins involved in free radical metabolism, heme oxygenase 1 and superoxide dismutase 3, whose expression was reduced in the hippocampus of water maze-trained animals. Besides their role in oxidative stress, these enzymes may be implicated in other physiological roles such as learning and memory. Indeed, impaired spatial memory is found in mice overexpressing these two proteins [57,58].

(iv) Transcription or Translation Regulation. Among the group of differentially expressed genes involved in transcription or translation regulation is the up-regulated gene encoding for cyclin Ania-6a, whose splicing is dynamically controlled by different forms of neuronal stimulation [59], and Jun-B, which is induced after different memory tasks [60].

The data presented here reveal distinct temporal gene expression profiles associated with learning and memory and demonstrate the utility of the cDNA microarray system as a means of dissecting the molecular basis of associative memory. It should be emphasized that the microarray provides estimates of changes in mRNA levels that are not necessarily correlated with the amount and function of the gene products. Protein turnover, and translation and posttranslational modifications of many gene products, may have dramatic effects on function that cannot be inferred from expression analysis alone. Nevertheless, the approach of the present invention provides information on the gene expression changes that occur during learning and memory, and identifies molecular targets and pathways whose modulation may allow new therapeutic approaches for improving cognition. As shown in previous studies, and in the present study for FGF-18, pharmacological or genetic modulation of these pathways can be effective in facilitating learning and memory.

As used herein, the term “pharmaceutically acceptable carrier” means a chemical composition with which the active ingredient may be combined and which, following the combination, can be used to administer the active ingredient to a subject. As used herein, the term “physiologically acceptable” ester or salt means an ester or salt form of the active ingredient which is compatible with any other ingredients of the pharmaceutical composition, which is not deleterious to the subject to which the composition is to be administered.

As used herein, “an effective amount” is an amount sufficient to produce an enhancement in memory, attentive cognition or learning, or increase FGF-18 levels in the subject's brain or hippocampus. As used herein, “enhancement in memory, attentive cognition or learning” refers to an improvement in memory, attentive cognition or learning as compared to a control subject or the subject prior to treatment. An improvement in memory, attentive cognition, or learning may be monitored by any number of clinical or biochemical tests or markers known to the skilled artisan.

As used herein, the term “subject” means a mammal.

As used herein, “pharmaceutically acceptable carrier” also includes, but is not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, and other mammals.

Pharmaceutical compositions that are useful in the methods of the invention may be prepared, packaged, or sold in formulations suitable for oral, parenteral, pulmonary, intranasal, buccal, or another route of administration. Other contemplated formulations include projected nanoparticles, liposomal preparations, resealed erythrocytes containing the active ingredient, and immunologically-based formulations. A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. In addition to the active ingredient, a pharmaceutical composition of the invention may further comprise one or more additional pharmaceutically active agents. Particularly contemplated additional agents include anti-emetics and scavengers such as cyanide and cyanate scavengers. Controlled- or sustained-release formulations of a pharmaceutical composition of the invention may be made using conventional technology.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets (each incorporated herein by reference). Tablets may fuirther comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin. Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

As used herein, “administration” of a composition includes any route of administration. Parenteral administration, for example, includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal injection, and kidney dialytic infusion techniques.

Typically dosages of the compound of the invention which may be administered to an animal, preferably a human, range in amount from 1 microgram to about 100 grams per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the memory, attention or learning deficit being treated, the type and age of the animal, etc.

The composition may be prepared and formulated by methods known in the art to enhance the uptake and transport of the compositions of the present invention. These methods include, but are not limited to, the formation of cholesteryl esters and other physiologically acceptable esters and conjugates of FGF-18 and/or packaging of the compositions and/or esters and conjugates into liposomes and artificial low density lipoproteins as described in U.S. Provisional Application Serial No. 60/430,476, incorporated by reference herein in its entirety.

### EXAMPLES

RNA preparation, microarray analyzes, quantitative RT-PCR and pharmacological studies were performed in a double-blind manner.

### Example 1

### Water Maze Learning

The subjects were 36 adult, male Wistar rats, each weighing 200-300 g. Rats were given access to food and water, and were maintained on a 12:12 light/dark cycle in a constant temperature (23° C.). Behavioral tests were performed as previously described [61], carried out in the light phase, and were in accordance with National Institutes of Health guidelines. To reduce stress in the experimental day, the first day was dedicated to swimming training, in the absence of an island. Each rat was placed in the pool for 2 min. and was returned to its home cage. In the next day, half of the rats were placed again in the pool for a 2.5-min swimming session and were used as swimming controls. The other half were given four consecutive trials to locate the platform, each trial lasting up to 2 min. Rats were required to spend 30 sec of an inter-trial interval on the platform. The rats' escape latency was measured by using a HVS2020 video tracking system (HVS Image, San Diego, Calif.). One, 6, and 24 h after training, swimming control and water maze-trained rats were killed and their hippocampi were rapidly dissected and frozen on dry ice. To verify that the rats that were used had indeed learned the spatial location of the island, a set of six rats was trained to find the island, and 24 h later they were tested on a quadrant analysis.

### Example 2

### Microarray Analysis

Use of the Affymetrix GeneChip™ Rat Neurobiology U34 array (Affymetrix, Santa Clara, Calif.), in connection with real-time PCR, has been previously described [62]. Hippocampal RNA from untrained animals (naïve), swimming control, and water maze-trained individual animals was extracted. Total RNA samples from each experimental condition were pooled into two groups, reverse transcribed, biotinylated, and hybridized to two Rat Neurobiology U34 arrays with the protocol outlined in the Gene Chip™ Expression Analysis Technical Manual (Affymetrix, Santa Clara, Calif.). The arrays were washed and stained by using a fluidics system with streptavidin-phycoerythrin (Molecular Probes Inc., Eugene, Oreg.), amplified with biotinylated anti-streptavidin antibody (Vector Laboratories, Burlingame, Calif.), and the scanned with a GeneArray™ Scanner (Affymetrix). To determine the quality of labeled targets before analysis on GeneChip™ Rat Neuorobiology U34 arrays, each sample was hybridized to one GeneChip™ “test 3” array. The image data were analyzed with the MICROARRAY SUITET™ 4.0 gene expression analysis program (Affymetrix). Normalization, filtering, and cluster analysis of the data were performed with the GENESPRING™ 4.2 software (Silicon Genetics, Redwood City, Calif.).

The raw data from each array were normalized as follows: Each measurement for each gene was divided by the 50^{th} percentile of all measurements. Each gene was then normalized to itself by making a synthetic positive control for that gene, and dividing all measurements for that gene by this positive control. This synthetic control was the median of the gene's expression values over all of the samples. Average difference values of less than zero represent probe sets where the intensity of the mismatched probe is, on average, greater than the perfect matched probe and; thus, the probe set is performing poorly. For this reason, normalized values below 0 were set to 0. Data derived from replicates (n=2) in experimental groups were used to perform pair-wise comparisons. An average fold change, derived from all possible pair-wise comparisons, greater than 2 and at least one raw average difference value above 100 was used as the cutoff for significant differences in gene expression.

### Example 3

### Real-Time Quantitative RT-PCR

To further confirm the reliability of the array data, the mRNA levels of 15 genes were quantified by real-time quantitative RT-PCR. Aliquots of cDNA (0.1 and 0.2 μg) from naïve, swimming control, and water maze-trained rats (six animals per group), and known amounts of external standard (purified PCR product, 10² to 10⁸ copies) were amplified in parallel reactions using specific primers. PCR amplifications were performed as described [63, 64]. Specificity of PCR products obtained was characterized by melting curve analysis, followed by gel electrophoresis and DNA sequencing.

### Example 4

### Behavioral Pharmacology

Thirteen male Wistar rats (250-300 g) were implanted stereotaxically with stainless steel guide cannulae in the right and left lateral ventricles (AP, −0.80 mm; Marc Levoy, 1.5 mm; DV, 3.6 mm) [65]. On day 1, one week after surgery, animals were subjected to a 2-min swimming training session. A water maze training session was then performed on days 2 and 3, which measured the ability of the animals to find a submerged platform to escape from the water. Two trails were given to each animal for each session. The escape latency and distance to find the platform were monitored as described above. Ten minutes after the second trial on day 2, an intracerebroventricular administration of drug or vehicle was performed in both lateral ventricles by introducing stainless steel injection cannulae into the implanted guide cannulate. Each injection cannula was connected to a 25-μl Hamilton syringe fastened onto a pump through polyethylene tubing filled with distilled water. Infusions were performed at a rate of 2 μl/min for 1 min in each side. Six animals received 0.94 pmol of FGF-18 (PeproTech Inc., Rocky Hill, N.J.) and the other seven received a control injection of vehicle (saline). Results are summarized in Table 1.

### References

Each of the references cited below are incorporated herein, in their entirety, by reference.

1 Byme, J. H. et al., in Advances in Second Messenger and Phosphoprotein Research Shenolikar, S. & Naim. A. C. (eds.) 47-107 (1993); Chetkovich, D. M. et al., Proc. Natl. Acad. Sci. U.S.A. 88:6467-6471 (1991); Ghirardi, M. et al., Neuron 9:479-489 (1992); Davis, R. L., Physiological Reviews 76:299-317 (1996); Hawkins, R. D. et al., Ann. Rev. Neurosci. 16:625-665 (1993).

2 Davis, H. P., Squire, L. R., Psychol. Bull. 96:518-559 (1984); Montarolo, P. G., et al., Science 234:1249-1254 (1986); Tully, T. et al., Cell 79:3 5-47 (1994); Schacher, S. et. Al., Science 240:1667-1669 (1988); Bailey, C. H., Kandel, E. R., Ann. Rev. Physiol. 55, 397-426 (1993).

3 J. Martinex, B. Derrick, Annu. Rev. Physchol. 47:173-203 (1996).

4 Hu et al., “Human fibroblast growth factor-18 stimulates fibroblast cell proliferation and is mapped to chromosome 14p11,” Oncogene, 18 (16): 2635-42 (1999).

5 Hu et al., “FGF-18, a novel member of the fibroblast growth factor family, stimulates hepatic and intestinal proliferation.” Molecular Cellular Biology, 18(10):6063-6074 (1998).

6 Robert A. Meyers, ed., Molecular Biology and Biotechnology: A Comprehensive Desk Reference, 121-122 (VCH Publishers 1995)).

7 Jack, Epilepsia 35:S21-S29 (1994).

8 Sapolsky, Behav. Brain Res. 57:175-182 (1993).

9 Mody et al. “Genome-wide gene expression profiles of the developing mouse hippocampus,” Proc. Natl. Acad. Sci. U.S.A. 98(15):8862-8867 (2201).

10 M. Kaser et al., U.S. Pat. No. 6,222,027 (2001).

11 R. Morris et al., Quart. J. Exp. Psychol. 38B:365-395 (1986).

12 See, e.g. Schena et al. (1995), “Quantitative monitoring of gene expression patterns with a complementary DNA micro-array,” Science 270:467-470; Lockhart et al. (1996), “Expression monitoring by hybridization to high-density oligonucleotide arrays,” Nature Biotechnology 14:1675-1680; Blanchard et al. (1996), “Sequence to array: Probing the genome's secrets,” Nature Biotechnology 14:1649; Ashby et al., “Methods for Drug Screening,” U.S. Pat. No. 5,569,588 (1996); Chen et al., “Ratio-based decisions and the quantitative analysis of cDNA micro-array images,” U.S. Pat. No. 6,245,517 (2001).

13 Leil, T. A., Ossadtchi, A., Cortes, J. S., Leahy, R. M., Smith, D. J. “Finding new candidate genes for learning and memory.” J. Neurosci Res 68:127-137 (2002).

14 S. Cavallero et al., Proc. Natl. Acad. Sci. USA, 94:9669-9673 (1997).

15 A. J. Silva, R. Paylor, J. M. Wehner, S. Tonegawa, Science 257, 206-11 (1992).

16 S. G. Grant et al., Science 258m 1903-10 (1992).

17 J. Z. Tsien, P. T. Huerta, S. Tonegawa, Cell 87, 1327-38 (1996).

18 K. Sakimura et al., Nature 373, 151-155 (1995).

19 Neural Cell Culture: A Practical Approach; Cohen, J. and Wilken, G., (Eds.) IRL Press at Oxford University Press, Oxford UK (1996)

20 N. Ohbayahshi et al., J. Biol. Chem. 273:18161-18164 (1998).

21 E. Lee et al., Clin. J. Physiol. 39:197-203 (1996)

22 D. Mott, D. Lewis, Int. Rev. Neurobiol. 36:97-223 (1994).

23 G. Calo et al., Br. J. Pharmacol. 129:1261-1283 (2000).

24R. Reinscheid, O. Civelli, Neuropeptides 36:72-76 (2002).

25 A. de Mendonca, J. Ribeiro, Neuroscience 62:385-390 (1994).

26 C. R. Park, Neurosci. Biobehav. Rev. 25:311-323 (2001).

27 T. A. Smith, Br. J. Biomed. Sci. 58:111-121 (2001).

28 D. R. Madden, Nat. Rev. Neurosci. 3:91-101 (2002).

29 S. Choc. Nat. Rev. Neurosci. 3:115-121 (2002).

30 M. Gomez et al., Neuro 30:241-248 (2001).

31 J. Masson et al., Pharmacol. Rev. 51:439-464 (1999).

32 K. Yamada et al., Br. J. Pharmacol. 115:852-858 (1995).

33 P. Chapman et al., NeuroReport 3:567-570 (1992).

34 L. Estall et al., Pharmacol. Biochem. Behav. 46:959-963 (1993).

35 A. Katzoff et al., J. Neurosci. 22:9581-94 (2002).

36 J. Komhauser, M. Greenberg, Neuron 18:839-842 (1997).

37 S. Impey et al., Neuro 23:11-14 (1999).

38 X. Zhen et al., J. Neurosci. 21:5513-5519 (2001).

39 R. J. Davis, J. Biol. Chem. 268:14533-14556 (1993).

40 D. Berman et al., J. Neurosci. 18:10037-10044 (1998).

41 M. Mayford et al., Science 274:1678-1683 (1996).

42 B. Xiao et al., Curr. Opin. Neurobiol. 10:370-374 (2000).

43 W. Zhang, et al., J. Neurosci. 19:96-108 (1999).

44 R. Jahn, T. Sudhof, Ann. Rev. Biochem. 68:863-911 (1999).

45 C. Ho et al., J. Comp. Neurol. 420:261-276 (2000).

46 M. Medina et al., Genomics 63:157-164 (2000).

47 K. Kosik et al., Proc. Natl. Acad. Sci. USA 81:7941-7945 (1984).

48 J. Leterrier, J. Eyer, J. Neurochem. 59:1126-1137 (1992).

49 W. Kaufmann et al., Neuropediatrics 26:109-113 (1995).

50 K. Fukunaga et al., Neurochem. Intl. 28:343-358 (1996).

51 H. Yamanouchi et al., Acta Neuropathol. 95:466-470 (1998).

52 N. Woolf et al., Brain Res. 821:241-249 (1999).

53 V. D'Agata et al., Neurobiol. Dis. 10:211-218 (2002).

54 The Dutch-Belgian Fragile X Consortium, Cell 78:23-33 (1994).

55 J. Wright et al., Peptides 23:221-246 (2002).

56 M. P. Mattson, W. Duan, J. Neurosci. Res. 58:152-166 (1999).

57 E. Gahtan et al., Eur. J. Neurosci. 10:538-544 (1998).

58 D. Morgan et al., Brian Res. 808:110-112 (1998).

59 J. Berke et al., Neuron 32:277-287 (2001).

60 W. Tischmeyer, R. Grimm, Cell. Mol. Life Sci. 55:564-574 (1999).

61 W. Zhao et al., Proc. Natl. Acad. Sci. U.S.A. 94:9669-9673 (1997).

62 G. Song. Et al., J. Neurosci. Res. 68:730-737 (2002).

63 S. Cavallaro et al., Eur. J. Neurosci. 13:1809-1815 (2001).

64 V. D'Agata et al., Neurobiol. Dis. 10:211-218 (2002).

65 C. Rachal Pugh et al., Neurosci. Biobehav. Rev. 25:29-41 (2001).

We claim:

1. A method of enhancing learning in an individual which comprises administering an effective amount of Fibroblast Growth Factor 18 (FGF-18).

2. A method of enhancing memory consolidation in an individual which comprises administering an effective amount of FGF-18.

3. A method of treating a condition selected from the group consisting of: impaired cognitive performance, learning deficit, cognition deficit, attention deficit, epilepsy, schizophrenia, Alzheimer's disease, and amnesiac syndromes, comprising the step of administering to an individual in need of such treatment a therapeutically effective amount of Fibroblast Growth Factor-18.

4. The method of claim 3, wherein the condition is impaired cognitive performance.

5. The method of claim 3, wherein the condition is a learning deficit.

6. The method of claim 3, wherein the condition is attention deficit.

7. The method of claim 3, wherein the condition is epilepsy.

8. The method of claim 3, wherein the condition is schizophrenia.

9. The method of claim 3, wherein the condition is Alzheimer's disease.

10. The method of claim 3, wherein the condition is an amnesiac syndrome.

11. A method for determining the susceptibility of a subject to a condition selected from the group consisting of: impaired cognitive performance, learning deficit, cognition deficit, attention deficit, epilepsy, schizophrenia, Alzheimer's disease and an amnesiac syndrome, wherein the method comprises the steps of:
(a) removing from the central nervous system of the subject a sample comprising Fibroblast Growth Factor-18 mRNA, and
(b) quantitating the Fibroblast Growth Factor-18 mRNA in said sample;
wherein the level of said Fibroblast Growth Factor-18 mRNA is indicative of said subject's susceptibility to said condition.

12. The method of claim 11, wherein the sample is obtained from the hippocampus.

13. A method for determining the pharmacological effect of a compound on the level of FGF-18 gene expression, comprising the steps of:
(a) growing one or more cultures of neural cells;
(b) measuring the level of FGF-18 gene expression in the cultured neural cells;
(c) contacting the compound with at least one of the cultures of neural cells; and
(d) measuring the level of FGF-18 gene expression in the cultured neural cells that have been contacted with the compound;
wherein a difference in the level of FGF-18 gene expression that correlates with exposure of the neural cells to the compound is indicative of a pharmacological effect of said compound.

14. A method for identifying memory-related proteins, comprising the steps of
(a) providing naïve, swimming control, and water-maze trained animals;
(b) extracting mRNA from the hippocampus of the naïve, control and trained animals;
(c) determining differential gene expression levels by quantitating and comparing mRNA levels in naïve, control and trained animals so as to identify “memory related genes”; and
(d) quantitating protein levels reflecting memory related genes for both control and target groups.

15. The method of claim 14, further comprising the step of validating the differentially expressed genes quantified in step (d) by quantitative RT-PCR.

16. The method of claim 15, wherein the quantitation of MRNA is carried out by a method selected from the group consisting of: Northern blotting, nuclease protection assays, array hybridization, RT-PCR, and hybridization with labeled oligonucleotide probes.

17. The method of claim 16, wherein the quantitation of mRNA is carried out by array hybridization.

18. A method of enhancing memory, attentive cognition or learning comprising the administration of a composition, wherein the composition comprises an effective amount of FGF-18 and a pharmaceutically acceptable carrier, to a subject in need thereof.

19. The method of claim 18, wherein the subject suffers from a condition selected from the group consisting of: impaired cognitive performance, learning deficit, cognition deficit, attention deficit, epilepsy, schizophrenia, Alzheimer's disease, and amnesiac syndromes.

20. The method of claim 18, wherein the composition is administered in an amount effective to increase FGF-18 levels in the subject's brain.

21. The method of claim 20, wherein the composition is administered in an amount effective to increase FGF-18 levels in the subject's hippocampus.
